(19) United States
(12) Patent Application Publication
(10) Pub. No.: US 2021/0130690 A1
(43) Pub. Date: May 6, 2021

(54) SEMICONDUCTOR NANOSTRUCTURES AND APPLICATIONS

(71) Applicant: YISSUM RESEARCH DEVELOPMENT COMPANY OF THE HEBREW UNIVERSITY OF JERUSALEM LTD, Jerusalem (IL)

(72) Inventors: Uri BANIN, Mevasseret Zion (IL); Botao JI, Jerusalem (IL)

(21) Appl. No.: 16/604,832

(22) PCT Filed: Apr. 16, 2018
(86) PCT No.: PCT/IL2018/050425

§ 371 Date: Oct. 11, 2019

Related U.S. Application Data
(60) Provisional application No. 62/487,039, filed on Apr. 19, 2017.

(51) Int. Cl. C09K 11/88

(52) U.S. Cl. CPC C09K 11/883; B82Y 20/00

(57) ABSTRACT

A colloidal nanostructure is provided associated with a heavy-metal-free semiconductor material.

### TECHNOLOGICAL FIELD

The invention generally concerns a method for the synthesis of novel Zn-based nanostructures.

### BACKGROUND

One dimensional semiconductor nanocrystals (quantum nanorods) exhibit great potentials in many applications including lasing, light-emitting diodes (LEDs) and solar cells, due to their unique optical and electronic properties. For example, CdSe/CdS dot-in-rod nanocrystals display linearly polarized emission and can be used to efficiently convert unpolarized backlight to white polarized light source for display applications. Nanorod-metal hybrids are shown to be good photocatalysts benefiting from light induced spatial charge separation at the rod-metal interface. However, many such studies mainly focus on semiconductor materials containing heavy metals, which are potentially toxic and environmentally restricted. Compared with cadmium chalcogenides nanorods, of which syntheses and properties have been well investigated, the syntheses of zinc chalcogenides nanorods are much more challenging, although they are more desirable for the toxic concerns and regulatory aspects. For example, among zinc chalcogenides, ZnTe with direct bang gap energy of 2.3 eV emerges as an attractive semiconductor for blue/green LEDs. Besides, the high conduction band edge position of ZnTe (−1.7 V) facilitates ultrafast charge transfer, which is extremely useful for energy applications. Zhang et al [1] developed a method to synthesize ZnTe nanorods with controllable aspect ratios by employing a highly reactive tellurium precursor. However, the obtained ZnTe nanorods showed no photoluminescence (PL) because of surface traps. Further modification of this nanostructure is necessary to make ZnTe nanorods fluorescent, which has not been achieved prior to this invention.

### PUBLICATIONS

[1] Zhang et al., J. Am. Chem. Soc., 2011, 133 (39), pp 15324-15327
[2] Oh et al., Science, 2017, 355, pp 616-619
[3] U.S. Pat. No. 7,394,094
[4] US Application No. 20150364645

### GENERAL DESCRIPTION

Herein, the inventors provide a family of Zn-based nanoparticles having an elongated central element, e.g., a rod structure, and a material deposited at each end, tip, of the elongated element. The nanostructures of the invention are free of heavy metals. In exemplary systems of the invention, a nanostructure comprised of ZnSe tips on both ends of a ZnTe nanorod is demonstrated, forming a heavy-metal-free ZnTe/ZnSe nanorod, also referred to herein as “nanodumbbell” (NDBs). While nanodumbells such as CdSe/Au, CdSe/PbSe, CdSe/CdTe, CdS/ZnSe and CdS/CdSe/ZnSe have been reported, such systems are unfavorable for various reasons, such as for containing Cd, which is a restricted element under the ROHS (Restriction of Hazardous Substances) directive of the European Union.

A particular interesting situation is achieved when the nanostructure of the invention is formed of two semiconductors that have type-II band offsets. In this configuration, the bands of the two semiconductors are staggered, where either the conduction band or valence band of one semiconductor is located in the band gap of a second semiconductor, the unique morphology of NDBs allows hole-electron charge separation into two different parts, which both directly touch the surroundings. This character makes these heavy-metal free NDBs ideal candidates for photo-catalysis and photovoltaic devices. Traditional type-II core/shell structures will trap one of the carriers in the core. Most recently, Oh et al [2] showed that double-heterojunction heavy-metal containing NDBs allowed both electroluminescence and photo-current generation upon light illumination. The fabricated LEDs from these NDBs were also responsive to external light and may open ways to many advanced display applications.

In a first aspect, the invention provides a heavy-metal-free zinc chalcogenide nanostructure, the nanostructure comprising an elongated element, each of the elongated element tips being coated with a second heavy-metal-free semiconductor material. In some embodiments, each of the element tips may be coated with the same or different semiconductor material.

As used herein, nanostructures of the invention are “heavy-metal free”; namely they do not contain any amount of a heavy metal, in either the material making up the elongated element or in the material(s) making up the tip coatings. In other words, the amount of the heavy metal in nanostructures of the invention is 0%. The heavy metals referred to may be selected from mercury, lead, cadmium and antimony. In some embodiments, the nanostructures are free of cadmium.

The heavy metal free nanostructures are colloidal nanostructures that comprise or consist at least one zinc chalcogenide material. As indicated, the nanostructures are structured of one or more elongated elements, each having one or two tips (or end regions or end tips) that are coated as defined herein. The tip(s) are the pointed end(s) of each elongated element. The one or more elongated elements, and in some embodiments also the tip(s) coating(s) comprise or consist a zinc chalcogenide material. Where both the one or more elongated elements and the tip(s) coating(s) comprise or consist a zinc chalcogenide material, the materials are different. For example, where the nanostructure comprises a single elongated element of a zinc chalcogenide material that is coated on both tip ends with a second different zinc chalcogenide material, each tip may be coated with the same material or different materials, such that a nanostructure may comprise two or more different zinc chalcogenide materials. In other words, nanostructures of the invention are heterostructures.

Thus the invention further provides a colloidal heavy-metal-free zinc chalcogenide nanostructure, the nanostructure comprising at least one elongated element of at least one zinc chalcogenide material, each of the at least one elongated elements having at least one tip ends coated with a heavy-metal-free semiconductor material, wherein the semiconductor material is different from the at least one zinc chalcogenide material.

While the elongated element is made of a zinc chalcogenide material, the tip coating may not be or may not comprise a zinc chalcogenide material.

The coating formed on the tip(s) of the elongated element is a monolayer or multilayered coating that is formed on the tip surface of the elongated element material. The coating does not cover the full circumference of the element, but only the tip apex region(s). The coating may increase the thickness (diameter) of the elongated element at the apex(es) and also the length (long axis) of the elongated element with the tip material. In embodiments where the nanostructure is a nanodumbbell (NDB), the length of the nanostructure (elongated element and tip coatings) is between 5 and 100 nm. In some embodiments, the average length of the NDBs, according to the invention, is between 5 and 90 nm, 5 and 80 nm, 5 and 70 nm, 5 and 60 nm, 5 and 50 nm, 5 and 40 nm, 5 and 30 nm, 5 and 20 nm, 10 and 90 nm, 10 and 80 nm, 10 and 70 nm, 10 and 60 nm, 10 and 50 nm, 10 and 40 nm, 10 and 30 nm or 10 and 20 nm. In some embodiments, the length is between 5 and 20 nm, 5 and 19 nm, 5 and 18 nm, 5 and 17 nm, 5 and 16 nm, 5 and 15 nm, 5 and 14 nm, 5 and 13 nm, 5 and 12 nm, 5 and 11 nm or 5 and l0 nm. In some embodiments, the length is between 6 and 20 nm, 6 and 19 nm, 6 and 18 nm, 6 and 17 nm, 6 and 16 nm, 10 and 20 nm, 10 and 19 nm, 10 and 18 nm, 10 and 17 nm, 10 and 16 nm, 10 and 15 nm, 15 and 20 nm, 15 and 25 nm, 15 and 30 nm or 15 and 35 nm.

In some embodiments, the length of each tip region formed upon coating of the elongated element apexes (as measured from one end of the region to its other along the long axis-length), independently of the other, is between 1 and 40% of the length of the elongated element prior to apex coating. In some embodiments, the length is between 1 and 39%, 1 and 38%, 1 and 37%, 1 and 36%, 1 and 35%, 1 and 34%, 1 and 33%, 1 and 32%, 1 and 31%, 1 and 30%, 1 and 29%, 1 and 28%, 1 and 27%, 1 and 26%, 1 and 25%, 1 and 24%, 1 and 23%, 1 and 22%, 1 and 21%, 1 and 20%, 1 and 19%, 1 and 18%, 1 and 17%, 1 and 16%, 1 and 15%, 1 and 14%, 1 and 13%, 1 and 12%, 1 and 11%, 1 and 10%, 1 and 9%, 1 and 8%, 1 and 7%, 1 and 6% or between 1 and 5%.

In some embodiments, the average length of the tip region formed upon coating of the elongated element apexes is between 0.5 and 5 nm. In some embodiments, the length is between 0.5 and 4.5 nm, 0.5 and 4 nm, 0.5 and 3.5 nm, 0.5 and 3 nm, 0.5 and 2.5 nm, 0.5 and 2 nm, 0.5 and 1.5 nm, 0.5 and 1 nm, 0.6 and 4.5 nm, 0.7 and 4.5 nm, 0.8 and 4.5 nm, 0.9 and 4.5 nm, 1 and 4.5 nm, 1.5 and 4.5 nm, 2 and 2.5 nm, 3 and 4.5 nm, 3.5 and 4.5 nm, 1 and 4 nm, 1 and 3.5 nm, 1 and 3 nm, 1 and 2.5 nm, 1 and 2 nm, 1 and 1.5 nm, 1.5 and 4.5 nm, 1.5 and 4 nm, 1.5 and 3.5 nm, 1.5 and 3 nm, 1.5 and 2.5 nm or 1.5 and 2 nm.

In some embodiments, the average thickness (diameter) of each tip region formed upon coating of the elongated element apexes, independently of the other, is between 0.5 and 5 nm. In some embodiments, the length is between 0.5 and 4.5 nm, 0.5 and 4 nm, 0.5 and 3.5 nm, 0.5 and 3 nm, 0.5 and 2.5 nm, 0.5 and 2 nm, 0.5 and 1.5 nm, 0.5 and 1 nm, 0.6 and 4.5 nm, 0.7 and 4.5 nm, 0.8 and 4.5 nm, 0.9 and 4.5 nm, 1 and 4.5 nm, 1.5 and 4.5 nm, 2 and 2.5 nm, 3 and 4.5 nm, 3.5 and 4.5 nm, 1 and 4 nm, 1 and 3.5 nm, 1 and 3 nm, 1 and 2.5 nm, 1 and 2 nm, 1 and 1.5 nm, 1.5 and 4.5 nm, 1.5 and 4 nm, 1.5 and 3.5 nm, 1.5 and 3 nm, 1.5 and 2.5 nm or 1.5 and 2 nm.

As shown herein, size histograms of exemplary nanoparticles of the invention provide an average lengths of NDBs to be 16.2 nm, with average tip widths of 6.3 nm, from which an average elongation of 2.1 nm along the nanorod axis.

The nanostructure may be of any shape comprising at least one elongated element. In some embodiments, the nanostructures comprise each a single elongated element, each having two end tips coated as defined herein. These may be regarded as nanorods or nanodumbells (NDB). Alternatively, the nanostructures may comprise two or more elongated elements, in which case they may be selected from angled (V-shaped) structures, or dipods, tripods, tetrapods, or higher structural homologs thereof. In such non-NDB nanostructures, each of the elongated structures may have a single end tip, to a total of end tips depending on the number of elongated elements and also on their structural connectivity.

As known in the art, a “chalcogenide material” is a material including a Group VI element, i.e., O, S, Se or Te. Thus, the zinc chalcogenide material is a material having at least one Group VI element. The zinc chalcogenide may be selected from ZnO, ZnS, ZnSe, ZnTe and alloys thereof.

In some embodiments, the nanostructure of the invention is a Type-II structure, wherein each electron and each positive hole are captured or confined in different semiconductor layers or different spatial positions. For example, in the ZnTe/ZnSe case, holes are confined in the elongated element material (ZnTe), whereas the electrons are localized in the tip shell material (ZnSe). Thus, the invention further provides a Type-II heavy-metal-free zinc-based nanostructure, the nanostructure comprising an elongated element of at least one zinc chalcogenide, each of the elongated element tips being coated with a zinc-chalcogenide semiconductor material.

Further provided is a Type-II heavy-metal-free zinc-based nanostructure, the nanostructure comprising an elongated element of at least one zinc chalcogenide, each of the elongated element tips being coated with a III-V semiconductor material.

In some embodiments, the III-V semiconductor material is selected from InAs, InP, GaAs, GaP, InN, GaN, InSb, GaSb, AlP, AlAs, AlSb and alloys such as InAsP, InGaAs.

In some embodiments, the nanostructures of the invention comprise an elongated element of a material selected from ZnTe, ZnSe, ZnS, ZnO and alloys thereof. In some embodiments, each of the element tips is coated with a material selected from ZnSe, ZnO, ZnS, ZnTe, InN, GaN, InP, GaP, AlP and alloys thereof. In some embodiments, the material is not ZnS The material of the elongated element and the material of either tip are not the same material.

In some embodiments, the nanostructures are of a material composition selected from ZnTe/ZnSe, ZnTe/InP, ZnSe/InP, ZnS/ZnSe, ZnS/ZnTe and ZnS/InP, wherein the first material, e.g., ZnTe in the case of ZnTe/ZnSe is the material of the elongated element, and ZnSe, in the same example, is the material of the tips.

In some embodiments, the nanostructures of the invention are constructed of an elongated element material and tip material(s) exhibiting tunable emission from ˜500 to ˜585 nm, providing means by which light emission from zinc chalcogenide nanorods may be tuned. Thus, the invention further provides a method of tuning light emission from a zinc chalcogenide nanorod (free of heavy metals), the method comprising forming or decorating the nanorod tips with a semiconductor material, as further detailed herein. In some embodiments, the amount of the semiconductor material formed or decorating the tips of the nanorod may be altered or modified or selected to permit tuning of the emission. As known in the art, the emission wavelength is determined by the valence band of elongated element material, e.g., ZnTe and the conduction band of tip material, e.g., ZnSe, the effective band gap energy, both depending on the size of each part (e.g., the diameter of the ZnTe region). As the size of the tip regions, e.g., ZnSe tips, decreases, the conduction band energy decreases, leading to a decrease in the effective band gap energy. As demonstrated below, comsol is used to predict the emission wavelength.

The invention further provides a nanodumbbel of a material selected from ZnTe/ZnSe, ZnTe/InP, ZnSe/InP, ZnS/ZnSe, ZnS/ZnTe and ZnS/InP.

The invention further provides a heavy-metal free nanodumbbell constructed of an elongated element consisting or comprising a zinc chalcogenide material, the elongated element having tip regions, each tip region comprising a coating of a semiconductor material; wherein the zinc chalcogenide material is selected from ZnTe, ZnSe and ZnS, and wherein the semiconductor material is selected from ZnSe, InP and ZnTe. In some embodiments, a combination of the zinc chalcogenide material and of the semiconductor material provides a Type-II structure. In some embodiments, the nanodumbells is of a material selected from ZnTe/ZnSe, ZnTe/InP, ZnSe/InP, ZnS/ZnSe, ZnS/ZnTe and ZnS/InP.

The invention further provides a device comprising a nanostructure according to the invention. The device may an electronic device, an optical device, an optoelectronic device, a device used in medicine, a device used in diagnosis, etc. In some embodiments, the device may be selected from displays, light conversion layer, back light unit, light emitting diode, and sensors.

Further provided is a method of preparing nanostructures according to the invention, the process comprising treating heavy-metal-free zinc chalcogenide nanostructure structurally comprising at least one elongated element with at least one precursor of a heavy-metal-free semiconductor material, under conditions permitting apex growth of the semiconductor material.

In some embodiments, the at least one precursor material is at least one metal precursor and at least one metal precursor and at least one chalcogenide or anion precursor. Where the heavy-metal-free semiconductor material is a zinc chalcogenide material, e.g., ZnSe, the at least one precursor is at least one zinc precursor and at least one chalcogenide precursor, e.g., precursor of Se.

In some embodiments, the metal precursor material is selected from the following:

Metal precursors as cations, wherein “M” represents a metal atom such as Zn, In, Ga, Al and others, include:
chlorides, e.g., selected from MCl, MCl₂, MCl₃, MCl₄, MCl₅, and MCl₆;
chlorides hydrates, e.g., selected from MCl.xH₂O, MCl₂.xH₂O, MCl₃.xH₂O, MCl₄.xH₂O, MCl₅.xH₂O, and MCl₆.xH₂O, wherein x varies based on the nature of M;
hypochlorites/chlorites/chlorates/cerchlorates (abbreviated ClOₙ⁻, n=1, 2, 3, 4), e.g., selected from MClOₙ, M(ClOₙ)₂, M(ClOₙ)₃, M(ClOₙ)₄, M(ClOₙ)₅, and M(ClOₙ)₆;
hypochlorites/chlorites/chlorates/perchlorates hydrates, e.g., selected from MClOₙ.xH₂O, M(ClOₙ)₂.xH₂O, M(ClOₙ)₃.xH₂O, M(ClOₙ)₄.xH₂O, M(ClOₙ)₅.xH₂O, and M(ClOₙ)₆.xH₂O, wherein x varies based on the nature of M, and n=1, 2, 3, 4;
carbonates, e.g., selected from M₂CO₃, MCO₃, M₂(CO₃)₃, M(CO₃)₂, M₂(CO₃)₂, M(CO₃)₃, M₃(CO₃)₄, M(CO₃)₅, M₂(CO₃)₇;
carbonate hydrates, e.g., selected from M₂CO₃.xH₂O, MCO₃.xH₂O, M₂(CO₃)₃.xH₂O, M(CO₃)₂.xH₂O, M₂(CO₃)₂.xH₂O, M(CO₃)₃.xH₂O, M₃(CO₃)₄.xH₂O, M(CO₃)₅.xH₂O, and M₂(CO₃)₇.xH₂O, wherein x varies based on the nature of M;
carboxylates (abbreviated RCO₂⁻, and including acetates), e.g., selected from MRCO₂, M(RCO₂)₂, M(RCO₂)₃, M(RCO₂)₄, M(RCO₂)₅, and M(RCO₂)₆;
carboxylates hydrates (abbreviated RCO₂⁻), e.g., selected from MRCO₂.xH₂O, M(RCO₂)₂.xH₂O, M(RCO₂)₃.xH₂O, M(RCO₂)₄.xH₂O, M(RCO₂)₅.xH₂O, and M(RCO₂)₆.xH₂O, wherein x varies based on the nature of M;
carboxylate (the group RCOO⁻, R is aliphatic chain, which may be saturated or unsaturated), e.g., selected from CH₃CH═CHCOOM (metal crotonate), CH₃(CH₂)₃CH═CH(CH₂)₇COOM (metal myristoleate), CH₃(CH₂)₅CH═CH(CH₂)₇COOM (metal palmitoleate), CH₃(CH₂)₈CH═CH(CH₂)₄COOM (metal sapienate), CH₃(CH₂)₇CH═CH(CH₂)₇COOM (metal oleate), CH₃(CH₂)₇CH═CH(CH₂)₇COOM (metal elaidate), CH₃(CH₂)₅CH═CH(CH₂)₉COOM (metal vaccinate), CH₃(CH₂)₇CH═CH(CH₂)₁₁COOM (metal erucate), C₁₇H₃₅COOM (metal stearate);
oxides, e.g., selected from M₂O, MO, M₂O₃, MO₂, M₂O₂, MO₃, M₃O₄, MO₅, and M₂O₇;
acetates, e.g., (the group CH₃COO⁻, abbreviated AcO⁻) selected from AcOM, AcO₂M, AcO₃M, and AcO₄M;
acetates hydrates, (the group CH₃COO⁻, abbreviated AcO⁻), e.g., selected from AcOM.xH₂O, AcO₂M.xH₂O, AcO₃M.xH₂O, and AcO₄M.xH₂O, wherein x varies based on the nature of M;
   acetylacetonates (the group C₂H₇CO₂⁻, abbreviated AcAc⁻), e.g., selected from AcAcM, AcAc₂M, AcAc₃M, and AcAc₄M;
   acetylacetonate hydrates (the group C₂H₇CO₂⁻, abbreviated AcAc), e.g., selected from AcAcM.xH₂O, AcAc₂M.xH₂O, AcAc₃M.xH₂O, and AcAc₄M.xH₂O, wherein x varies based on the nature of M;
nitrates, e.g., selected from MNO₃, M(NO₃)₂, M(NO₃)₃, M(NO₃)₄, M(NO₃)₅, and M(NO₃)₆;
nitrates hydrates, e.g., selected from MNO₃.xH₂O, M(NO₃)₂.xH₂O, M(NO₃)₃.xH₂O, M(NO₃)₄.xH₂O, M(NO₃)₅.xH₂O, and M(NO₃)₆.xH₂O, wherein x varies based on the nature of M;
nitrites, e.g., selected from MNO₂, M(NO₂)₂, M(NO₂)₃, M(NO₂)₄, M(NO₂)₅, and M(NO₂)₆;
nitrites hydrates, e.g., selected from MNO₂.xH₂O, M(NO₂)₂.xH₂O, M(NO₂)₃.xH₂O, M(NO₂)₄.xH₂O, M(NO₂)₅.xH₂O, and M(NO₂)₆.xH₂O, wherein x varies based on the nature of M;
cyanates, e.g., selected from MCN, M(CN)₂, M(CN)₃, M(CN)₄, M(CN)₅, M(CN)₆;
cyanates hydrates, e.g., selected from MCN.xH₂O, M(CN)₂.xH₂O, M(CN)₃.xH₂O, M(CN)₄.xH₂O, M(CN)₅.xH₂O, and M(CN)₆.xH₂O, wherein x varies based on the nature of M;
sulfides, e.g., selected from M₂S, MS, M₂S₃, MS₂, M₂S₂, MS₃, M₃S₄, MS₅, and M₂S₇;
sulfides hydrates, e.g., selected from M₂S.xH₂O, MS.xH₂O, M₂S₃.xH₂O, MS₂.xH₂O, M₂S₂.xH₂O, MS₃.xH₂O, M₃S₄.xH₂O, MS₅.xH₂O, and M₂S₇.xH₂O, wherein x varies based on the nature of M;
sulfites, e.g., selected from M₂SO₃, MSO₃, M₂(SO₃)₃, M(SO₃)₂, M₂(SO₃)₂, M(SO₃)₃, M₃(SO₃)₄, M(SO₃)₅, and M₂(SO₃)₇;
sulfites hydrates selected from M₂SO₃.xH₂O, MSO₃.xH₂O, M₂(SO₃)₃.xH₂O, M(SO₃)₂.xH₂O, M₂(SO₃)₂.xH₂O, M(SO₃)₃.xH₂O, M₃(SO₃)₄.xH₂O, M(SO₃)₅.xH₂O, and M₂(SO₃)₇.xH₂O, wherein x varies based on the nature of M;
hyposulfite, e.g., selected from M₂SO₂, MSO₂, M₂(SO₂)₃, M(SO₂)₂, M₂(SO₂)₂, M(SO₂)₃, M₃(SO₂)₄, M(SO₂)₅, and M₂(SO₂)₇;
hyposulfite hydrates, e.g., selected from M₂SO₂.xH₂O, MSO₂.xH₂O, M₂(SO₂)₃.xH₂O, M(SO₂)₂.xH₂O, M₂(SO₂)₂.xH₂O, M(SO₂)₃.xH₂O, M₃(SO₂)₄.xH₂O, M(SO₂)₅.xH₂O, and M₂(SO₂)₇.xH₂O, wherein x varies based on the nature of M;
sulfate, e.g., selected from M₂SO₃, MSO₃, M₂(SO₃)₃, M(SO₃)₂, M₂(SO₃)₂, M(SO₃)₃, M₃(SO₃)₄, M(SO₃)₅, and M₂(SO₃)₇;
sulfate hydrates, e.g., selected from M₂SO₃.xH₂O, MSO₃.xH₂O, M₂(SO₃)₃.xH₂O, M(SO₃)₂.xH₂O, M₂(SO₃)₂.xH₂O, M(SO₃)₃.xH₂O, M₃(SO₃)₄.xH₂O, M(SO₃)₅.xH₂O, and M₂(SO₃)₇.xH₂O, wherein x varies based on the nature of M;
thiosulfate, e.g., selected from M₂S₂O₃, MS₂O₃, M₂(S₂O₃)₃, M(S₂O₃)₂, M₂(S₂O₃)₂, M(S₂O₃)₃, M₃(S₂O₃)₄, M(S₂O₃)₅, and M₂(S₂O₃)₇;
thioulfate hydrates, e.g., selected from M₂S₂O₃.xH₂O, MS₂O₃.xH₂O, M₂(S₂O₃)₃.xH₂O, M(S₂O₃)₂.xH₂O, M₂(S₂O₃)₂.xH₂O, M(S₂O₃)₃.xH₂O, M₃(S₂O₃)₄.xH₂O, M(S₂O₃)₅.xH₂O, and M₂(S₂O₃)₇.xH₂O, wherein x varies based on the nature of M;
dithionites, e.g., selected from M₂S₂O₄, MS₂O₄, M₂(S₂O₄)₃, M(S₂O₄)₂, M₂(S₂O₄)₂, M(S₂O₄)₃, M₃(S₂O₄)₄, M(S₂O₄)₅, and M₂(S₂O₄)₇;
dithionites hydrates, e.g., selected from M₂S₂O₄.xH₂O, MS₂O₄.xH₂O, M₂(S₂O₄)₃.xH₂O, M(S₂O₄)₂.xH₂O, M₂(S₂O₄)₂.xH₂O, M(S₂O₄)₃.xH₂O, M₃(S₂O₄)₄.xH₂O, M(S₂O₄)₅.xH₂O, and M₂(S₂O₄)₇.xH₂O, wherein x varies based on the nature of M;
phosphates, e.g., selected from M₃PO₄, M₃(PO₄)₂, MPO₄, and M₄(PO₄)₃;
phosphates hydrates, e.g., selected from M₃PO₄.xH₂O, M₃(PO₄)₂.xH₂O, MPO₄.xH₂O, and M₄(PO₄)₃.xH₂O, wherein x varies based on the nature of M;
Metal alkyls;
Metal alkoxides;
Metal amines;
Metal phosphines;
Metal thiolates;
Combined cation-anion single source precursors, i.e., molecules that include both cation and anion atoms, for example of the formula M(E₂CNR₂)₂ (M=Pb, Rb, E=S, P, Se, Te, O, As, and R=alkyl, amine alkyl, silyl alkyl, phosphoryl alkyl, phosphyl alkyl).

In some embodiments, the chalcogenide or anion precursor may be an organic precursor of the chalcogenide metal (or the metal anion) or a halide precursor thereof.

In some embodiments, the tip material is a zinc chalcogenide and the at least one precursor is at least one zinc precursor and at least one chalcogenide precursor. The at least one zinc precursor is selected from the above metal precursors. In some embodiments, the zinc precursor is a zinc carboxylate, as defined herein, e.g., zinc oleate. In some embodiments, the chalcogenide atom precursor is an organic complex or form of the chalcogenide. In some embodiments, the precursor is TOP-chalcogenide.

In some embodiments, the metal precursor and the chalcogenide or anion precursor are added alternatively to a medium comprising the heavy-metal-free zinc chalcogenide nanostructure. In some embodiments, the heavy-metal-free zinc chalcogenide nanostructure is first treated with one of the at least one metal precursor and precursor of the chalcogenide or anion, and thereafter is treated with the other of the at least one metal precursor and precursor of the chalcogenide or anion.

In some embodiments, the heavy-metal-free zinc chalcogenide nanostructure is treated with the precursor(s) under conditions permitting material coating of the elongated element of the nanostructure. These conditions include one or more of the following:
1. Inert conditions (e.g., under inert gas, or oxygen free environment, or under vacuum);
2. A temperature between 100 and 300° C., between 100 and 250° C., between 200 and 300° C. or between 200 and 250° C.; and/or
3. Treating the medium comprising the elongated structure and at least one precursor with a chloride solution (e.g., a chloride-contained solution prepared for example from ZnCl₂ and additives such as tetradecylphosphonic acid (TDPA), oleylamine and TOP), optionally under UV irradiation or under suitable heating (e.g., a temperature between 100 and 300° C.).

In some embodiments, where the nanostructure of the invention is a heavy-metal free nanodumbbell constructed of a nanorod element (the elongated element) consisting or comprising a zinc chalcogenide material, and each tip region of the elongated element comprises a coating of a semiconductor material; and wherein the zinc chalcogenide material is selected from ZnTe, ZnSe and ZnS, and the semiconductor material is selected from ZnSe, InP and ZnTe; the method of the invention comprises treating nanorods of the zinc chalcogenide material with at least one precursor of the semiconductor material, the at least one precursor being at least one precursor of Zn, or In, and at least one precursor of Se, Te or P, at a temperature between 100 and 300° C.

In some embodiments, the nanorod elements are first treated with the at least one precursor of Zn, or In, and subsequently with the at least one precursor of Se, Te or P.

In some embodiments, the nanorod elements are first treated with the at least one precursor of Se, Te or P and subsequently with the at least one precursor of Zn, or In.

In some embodiments, the sequential treatment of the nanorods with the precursors is repeated one or more additional times so as to provide a coating of multiple material layers.

In some embodiments, the precursors are selected to provide a Type-II structure. In some embodiments, the nanodumbells produced are of a material selected from ZnTe/ZnSe, ZnTe/InP, ZnSe/InP, ZnS/ZnSe, ZnS/ZnTe and ZnS/InP.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-E provide (FIG. 1A) absorption evolution of ZnTe nanorods synthesis and (FIG. 1B) TEM image of ZnTe nanorods with an increasing rate of 5° C./minute; corresponding diameter and length histograms are shown in FIGS. 1C and 1D. FIG. 1E provides a TEM image of ZnTe nanorods with an increasing rate of 10° C./minute.

FIGS. 2A-D provide TEM images of ZnTe nanorods (FIG. 2A) and ZnTe/3ZnSe NDBs (FIG. 2B). (FIG. 2C) Normalized absorption spectra (Abs) and photoluminescence spectra (PL) of ZnTe nanorods and ZnTe/3ZnSe NDBs; No emission was observed from Bare ZnTe nanorods. (FIG. 2D) Schematic representation of band offsets in ZnTe/ZnSe NDBs presenting the indirect charge recombination; bulk values of band offsets of ZnTe and ZnSe are used.

FIGS. 3A-D provide TEM images of ZnTe/ZnSe NDBs by adding different amount s of ZnSe precursors and corresponding length histograms The average length is also shown. (FIG. 3A) 1 monolayer equivalent of ZnSe; (FIG. 3B) 2 monolayers equivalent of ZnSe; (FIG. 3C) 3 monolayers equivalent of ZnSe; (FIG. 3D) 4 monolayers equivalent of ZnSe.

FIGS. 4A-C depicts the evolution of (FIG. 4A) absorption and (FIG. 4B) emission spectra in an exemplary synthesis of “ZnTe/3ZnSe” NDBs; the zinc and selenium precursors with the calculated amounts for growing one complete shell on the existing nanoparticles were alternately added every 15 minutes at 240° C.; after the 3^{rd} addition of selenium precursor, only zinc precursor was added every 30 minutes to further promote the growth of ZnSe as well as the surface passivation. a) Bare ZnTe nanorods; b-d) 1, 2 and 3 monolayers of ZnSe; e and f) 2 and 4 more zinc additions. (FIG. 4C) Quantitative representation of PL wavelength and QY evolution as a function of reaction time in the same synthesis of ZnTe/3ZnSe NDBs.

FIGS. 5A-C follows ZnSe growth on ZnTe nanorods by injecting Selenium precursor (TOP-Se, for 6 monolayer of ZnSe) in Zn precursor solution (Zn oleate dissolved in the mixture of TOP and oleylamine) which contained ZnTe nanorods. The reaction temperature was 260° C. and the reaction time was 60 minutes. (FIG. 5A) Absorption and (FIG. 5B) emission spectra evolution and (FIG. 5C) TEM image of ZnTe/ZnSe nanoparticles at the end of synthesis. The quantum yield of the obtained nanoparticles is typically smaller than 5%. The NDBs structure could be recognized but less defined.

FIGS. 6A-E provide (FIG. 6A) XRD of ZnTe NRs (black) and ZnTe/ZnSe NDBs with increased amounts of ZnSe precursors: 2 (blue) and 4 (red) monolayers. The standard XRD stick-patterns of bulk wurtzite ZnTe and zinc blende ZnSe are also shown for comparison. High-resolution TEM (HRTEM) images of ZnTe nanorods (FIG. 6B) and ZnTe/3ZnSe NDBs (FIG. 6C). (FIG. 6E) Elemental analysis of Se (red line) and Te (blue line) (EDX spectra line scan, smoothed, see method part) along the long axis of a single ZnTe/3ZnSe NDB. Corresponding STEM image of the measured ZnSe/ZnTe NDBs is shown in (FIG. 6D) with the thick red line indicating the scan axis. All scale bars are equal to 5 nm.

FIGS. 7A-B provide (FIG. 7A) The energy dispersive X-ray (EDX) measurement of the ZnTe/ZnSe NDBs confirmed the existence of Zn (59%), Te (20%) and Se (20%). (FIG. 7B) HAADF-STEM image of ZnTe/ZnSe NDBs (the sample in FIG. 3C). The sample seemed not stable enough when elements line-scan or mapping was performed under STEM mode. No useful information was obtained.

FIGS. 8A-B provide (FIG. 8A) Te 3d and (FIG. 8B) Se 3d XPS spectra of ZnTe nanorods, ZnTe/ZnSe NDBs. The Te 3d spectra can be seen in both ZnTe nanorods and ZnTe/ZnSe NDBs. The relative intensity did not greatly decrease after the ZnSe growth, which can be explained by the formation of dumbbell structure. In the case of core/shell quantum dots, several layers of full shell growth significantly decrease or even completely block the signals from the core. The Se 3d spectra were detected in ZnTe/ZnSe NDBs.

FIG. 9 provides PL spectra of ZnTe with different amounts of ZnSe precursors.

FIGS. 10A-D provide (FIG. 10A) PL decay traces of ZnTe with different amounts of ZnSe precursors. (FIG. 10B) The calculated wave functions (electrons and holes distribution) for NDBs samples of ZnTe/1ZnSe and ZnTe/3ZnSe by effective-mass simulations. (FIG. 10C) Comparison of experimental (red triangles) and calculated PL emission energies (blue circles) of ZnTe/ZnSe NDBs as a function of ZnSe tip width along the c-axis of the ZnTe nanorod. An aspect ratio of 3 between the widths along and perpendicular to the c-axis of ZnTe nanorod is used in the calculations based on the measured values. (FIG. 10D) Comparison of the calculated relative exciton overlap integrals (blue circles) of ZnTe/ZnSe NDBs as a function of the ZnSe tip width with the overlap integral of ZnTe/1ZnSe NDBs is used as the reference. Experimental relative radiative transition rates are marked as red triangles.

FIGS. 11A-B provide PL wavelength (FIG. 11A) and quantum yield (FIG. 11B) evolution of ZnTe/3ZnSe NDBs with different zinc treatments after the 3^{rd} injection of selenium precursor. Compared to the case with no more zinc precursor addition, adding more zinc oleate induced larger red shift and higher quantum yield. However, when ZnCl₂-TDPA solution was introduced, the PL wavelength did not shift to the red any more, accompanied by the quantum yield enhancement.

FIG. 12 depicts evolution of Quantum yield for ZnTe/ZnSe NDBs with different zinc treatments for the last two injections of zinc precursor. Compared to zinc oleate, the obtained ZnTe/ZnSe NDBs with the addition of ZnCl₂ and ZnCl₂-TDPA displayed significantly enhanced quantum yield. All three samples had similar emission wavelength.

FIGS. 13A-B provide (FIG. 13A) Absorption and emission spectra (FIG. 13B) TEM image of ZnTe/3ZnSe NDBs with chloride treatment.

FIGS. 14A-D show optimization of NDBs optical properties by ZnCl₂ surface treatment. (FIG. 14A) High resolution Cl 2p XPS spectra with fits for Cl 2p_{3/2} (197.0 eV) and Cl 2p_{1/2} (198.6 eV). (FIG. 14B) PL decay traces of ZnTe/ZnSe NDBs without ZnCl₂ treatment (black) and with ZnCl₂ treatment (red). (FIGS. 14C and D) Comparison of PL QY and PL wavelength without ZnCl₂ treatment (black) and with ZnCl₂ treatment (red). The inset in (FIG. 14D) shows optical images of various ZnTe/ZnSe NDBs samples with chloride treatment under UV illumination.

FIGS. 15A-B provide (FIG. 15A) PLE photo-selection measurements and corresponding fluorescence anisotropy (FIG. 15B) of ZnTe/ZnSe NDBs.

### DETAILED DESCRIPTION OF EMBODIMENTS

Materials. Zinc acetate (anhydrous, 99.99%), zinc oxide (ZnO, 99.0%), 1-octadecene (ODE, 90%), oleic acid (OA, 90%), tellurium (shot, 1-2 mm, 99.999%), superhydride solution (lithium triethylborohydride in tetrahydrofuran, 1.0 M), selenium (99.99%), oleylamine (OLA, 70%), zinc chloride (99.999%) were purchased from Sigma. Trioctylphosphine (TOP, 97%) was purchased from Strem. Tetradecylphosphonic acid (TDPA, 99%) was purchased from PCI synthesis. All chemicals were used as received without any further purification. It should be noted that all the manipulations in this report were performed under inert atmosphere in the glove box filled with nitrogen or Schlenk line.

Preparation of precursors. Trioctylphosphine-tellurium (TOP-Te, 1.0 M) was prepared by dissolving Te shot in TOP in a glovebox. Selenium stock solution. Trioctylphosphine-selenium (TOP-Se, 0.1 M) was prepared by dissolving selenium powder in TOP in glovebox. Zinc stock solution. A solution of zinc oleate (Zn(OA)₂, 0.1 M) in TOP was synthesized by heating 0.833 g (10.23 mmol) of zinc oxide in 20.4 mL of oleic acid and 80 mL of TOP at 300° C. under argon until a colorless solution was obtained. A ZnCl₂ solution (0.1 M) for the chloride treatment was prepared by heating 0.545 g of ZnCl₂ (4 mmol) in the mixture of oleylamine (20 mL) and TOP (20 mL) at 150° C. for 30 minutes under vacuum. Another ZnCl₂ solution contained TDPA was prepared by the same procedure with the addition of 0.557 g of TDPA (2 mmol). All the precursor solutions were stored in the glovebox.

Synthesis of ZnTe nanorods. 520 mg of zinc acetate (2.8 mmol) were loaded in a 150 mL three-neck flask which contained 8.0 mL of oleic acid and 40.0 mL of ODE. The flask was degassed at 90° C. for 2 hours until a clear solution was obtained. Under argon, the solution was heated to 200° C. first and then cooled down to 160° C. In the glove box, 3.2 mL of superhydride solution (1.0 M in THF) were added into 2.0 mL of fresh TOP-Te solution (1.0 M), followed by the addition of 8.0 mL oleylamine under stirring. This dark purple tellurium precursor solution was taken out of glove box and immediately injected into the flask at 160° C. under vigorous stirring. The reaction temperature was then increased to 240° C. at a rate of 5° C./minute. In this process, tetrahydrofuran in the flask was removed through a syringe to avoid violent boiling. The reaction was kept at 240° C. for 50 minutes before cooling down. The flask was transferred to the glove box and 25.0 mL of dry toluene were added to the flask.

ZnSe growth on ZnTe nanorods. ZnTe nanorods were purified by centrifugation using hexane/ethanol as the solvent/anti-solvent system for three times and redispersed in hexane. The molar absorptivity at 350 nm was measured and used to calculate the concentration of ZnTe nanorods according to literature method. ˜10 nmol of ZnTe nanorods were introduced to a 25 mL three-neck flask with 1.25 mL of TOP and 0.75 mL of oleylamine The flask was degassed under vacuum at 90° C. for one hour to remove solvents with low boiling points. For the growth of ZnSe, a layer-by-layer synthesis method was applied. The temperature was increased to 240° C. under argon. Zinc and selenium precursors with the calculated amounts for growing one complete shell on the existing nanoparticles were alternately added every 15 minutes. To be specific, for example in a typical synthesis of ZnTe/ZnSe NDBs with the addition of ZnSe precursors for 3 monolayers, 0.16 mL of the zinc stock solution (zinc oleate in TOP, 0.1 M) was injected dropwise. The same amount of selenium stock solution (TOP-Se, 0.1 M) was added after 15 minutes. 0.19 mL and 0.22 mL of zinc and selenium stock solutions were then successively injected every 15 minutes. This was followed by adding 0.29 mL of zinc stock solution and waiting for 30 minutes. In order to promote the reaction between selenium and zinc and to improve the surface passivation, 0.34, 0.38, 0.43 and 0.48 mL of zinc stock solution were added every 30 minutes (see Table 1). One time addition of all zinc stock solutions (1.63 mL) and waiting for 2 hours gave similar results (in terms of emission wavelength and quantum yield). Aliquots were taken to monitor the synthesis progress. Similarly, different calculated amounts of ZnSe precursors were added to tune the size of the ZnSe tips, also with additional zinc stock solution being injected. For the optimization of optical properties, the last two addition of zinc stock solution (0.43 and 0.48 mL) in the above synthesis were replaced by ZnCl₂ solution. The final product was precipitated by adding ethanol, centrifuged, and redispersed in hexane.

TABLE 1
Details for the synthesis of ZnTe/ZnSe NDBs with different amounts of ZnSe precursors. 10.0 nmol of ZnTe nanorods
were dispersed in a three-neck flask with 1.25 mL of TOP and 0.75 mL of oleylamine. The solution was heated
to 240° C. under argon. The zinc precursor (zinc stock solution) was zinc oleate (0.1M). The selenium
precursor (selenium stock solution) was TOP-Se (0.1M). ‘t = 0 min’ meant the beginning of the reaction.

| Injection | | | | |
|---|---|---|---|---|
| number | ZnTe/1ZnSe | ZnTe/2ZnSe | ZnTe/3ZnSe | ZnTe/4ZnSe |
| 1 | 0.16 mL (Zn, t = 0 min) | 0.16 mL (Zn, t = 0 min) | 0.16 mL (Zn, t = 0 min) | 0.16 mL (Zn, t = 0 min) |
| 2 | 0.16 mL (Se, t = 15 min) | 0.16 mL (Se, t = 15 min) | 0.16 mL (Se, t = 15 min) | 0.16 mL (Se, t = 15 min) |
| 3 | 0.21 mL (Zn, t = 30 min) | 0.19 mL (Zn, t = 30 min) | 0.19 mL (Zn, t = 30 min) | 0.19 mL (Zn, t = 30 min) |
| 4 | 0.25 mL (Zn, t = 60 min) | 0.19 mL (Se, t = 45 min) | 0.19 mL (Se, t = 45 min) | 0.19 mL (Se, t = 45 min) |
| 5 | 0.29 mL (Zn, t = 90 min) | 0.25 mL (Zn, t = 60 min) | 0.22 mL (Zn, t = 60 min) | 0.22 mL (Zn, t = 60 min) |
| 6 | 0.34 mL (Zn, t = 120 min) | 0.29 mL (Zn, t = 90 min) | 0.22 mL (Se, t = 75 min) | 0.22 mL (Se, t = 75 min) |
| 7 | 0.38 mL (Zn, t = 150 min) | 0.34 mL (Zn, t = 120 min) | 0.29 mL (Zn, t = 90 min) | 0.25 mL (Zn, t = 90 min) |
| 8 | | 0.38 mL (Zn, t = 150 min) | 0.34 mL (Zn, t = 120 min) | 0.25 mL (Se, t = 105 min) |
| 9 | | 0.43 mL (Zn, t = 180 min) | 0.38 mL (Zn, t = 150 min) | 0.34 mL (Zn, t = 120 min) |
| 10 | | | 0.43 mL (Zn, t = 180 min) | 0.38 mL (Zn, t = 150 min) |
| 11 | | | 0.48 mL (Zn, t = 210 min) | 0.43 mL (Zn, t = 180 min) |
| 12 | | | | 0.48 mL (Zn, t = 210 min) |
| 13 | | | | 0.54 mL (Zn, t = 240 min) |

Characterization. The samples were sealed in a cuvette for all the optical measurements. UV-vis absorption and emission spectra were recorded on a JASCO V-570 spectrometer and Varian Cary Eclipse spectrophotometer, respectively. Fluorescence lifetime and photo-selection excitation measurements were performed on Edinburgh Instruments FLS920 fluorometer with a TCC900 TCSPC (time correlated single photon counting) card. X-ray diffraction (XRD) measurements were performed on a Phillips PW1830/40 diffractometer using the Cu Kα photons. Transmission electron microscopy (TEM), High-resolution TEM (HRTEM), scan TEM (STEM) images and energy dispersive X-ray (EDX) spectra were obtained on FEI Tecnai F20 G² HRTEM with a field-emission gun as an electron source. X-ray photoelectron spectroscopy (XPS) measurements were performed on a Kratos AXIS Ultra X-ray photoelectron spectrometer. Inductively coupled plasma mass spectrometry (ICP-MS) measurements were carried out using a Perkin-Elmer Optima 3000.

### Results and Discussions

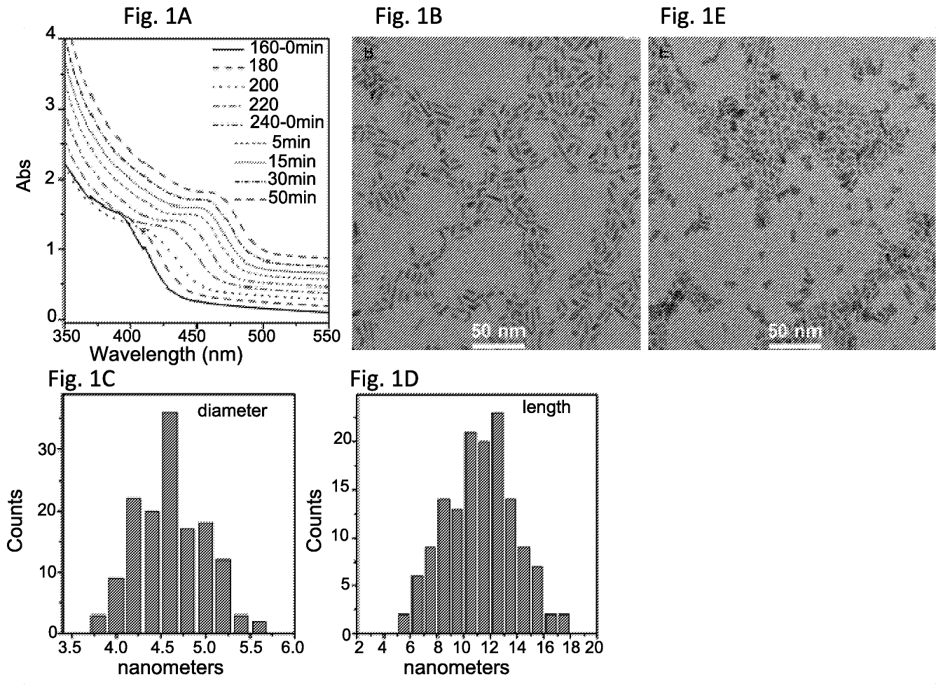

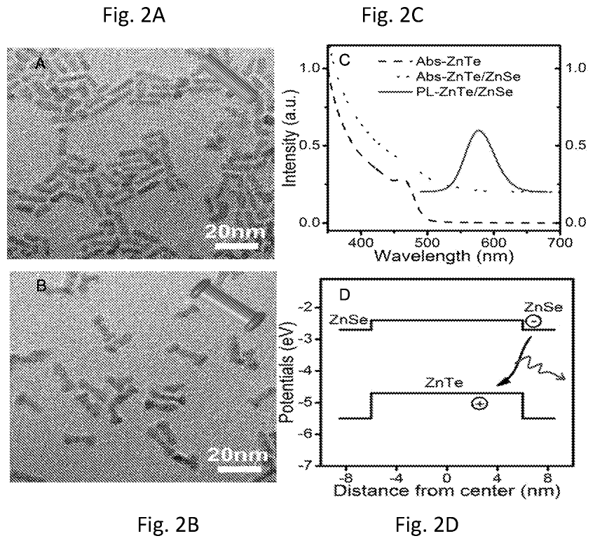

ZnTe nanorods were first synthesized according to a published method with minor modifications. A highly reactive polytellurides solution, which was prepared by mixing superhydride solution and TOP-Te, was injected into zinc oleate solution at 160° C. The temperature was increased to 240° C. at a rate of 5° C./minute. Relatively mono-dispersed ZnTe nanorods with diameter of 4.6 nm and length of 12 nm are obtained after 50 minutes of growth at 240° C. as shown in FIG. 2A. The absorption spectra exhibit the excitonic peak around 463 nm (FIG. 2C), indicating a narrow diameter dispersion. An increasing rate of 10° C./minute as in the literature results in the formation of poorly-defined ZnTe nanorods (FIG. 1). The highly reactive polytellurides contain a mixture of reduced Te species including Te²⁻, Te₂²⁻ and Te₃²⁻ which have different reactivity. The most reactive Te⁻² ions react with zinc precursor and nucleate in wurtzite phase at low temperature (160° C.). The rest reduced Te species then react at elevated temperature and grow on specific facets and eventually form ZnTe nanorods. Too fast heating speed may destroy the growth balance and lead to the growth of irregular shaped nanoparticles. The synthesized ZnTe nanorods do not show any photoluminescence (PL), consistent with previous reports. The absence of fluorescence presumably results from surface trap states of colloidal ZnTe nanoparticles because of its frangibility to oxidation. Exposing purified ZnTe nanorods (dispersed in unpolar organic solvent) in air for several hours leads to the appearance of black precipitate, indicating the production of Te from the oxidation of ZnTe. Thereby, all the manipulations in this work were performed strictly under conditions free of oxygen and water to avoid any possible oxidation.

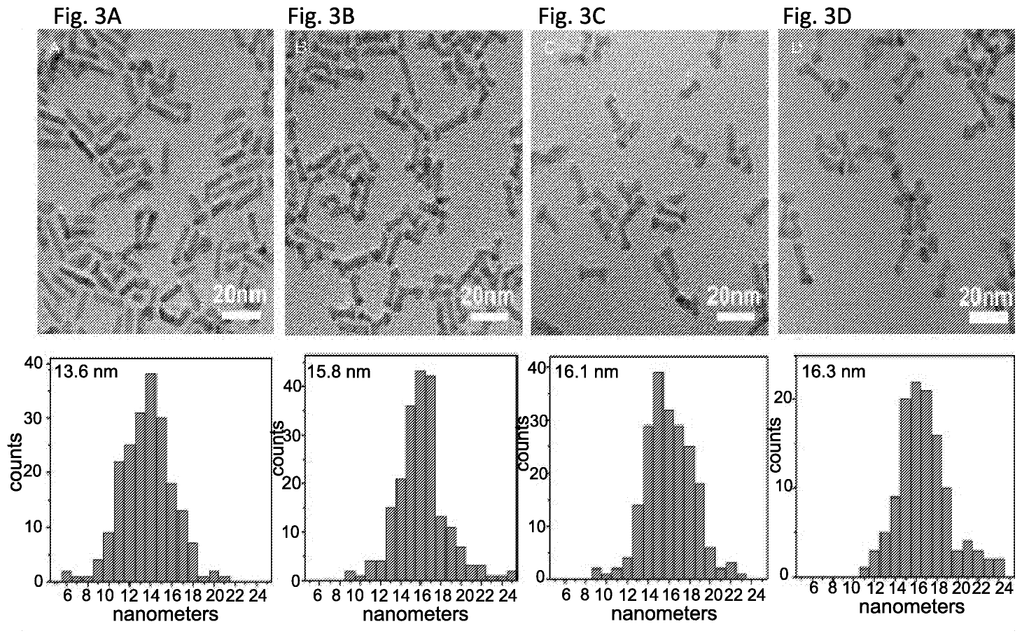

The growth of ZnSe tips on ZnTe nanorods was performed via a layer-by-layer method in which suitable calculated amounts of Zn and Se precursors are added sequentially. The obtained ZnTe nanorods were used for the synthesis of ZnTe/ZnSe NDBs through the tip growth of ZnSe. Carboxylate acid and phosphoric acid are avoided to use because they are too corrosive and will cause decomposition of ZnTe. Purified ZnTe nanorods were dispersed in the mixture of TOP and oleylamine (OAm). Zinc and selenium precursors with the calculated amounts for growing one complete shell on the existing nanoparticles were alternately added every 15 minutes at 240° C. under Ar. When the sequential additions of ZnSe precursors with desired amounts were completed, more zinc stock solution was injected at 240° C. to promote selenium reacting with zinc and improve the surface passivation (see details in experimental parts). Hereafter, ZnTe/nZnSe were used to represent ZnTe/ZnSe NDBs with the addition of ZnSe precursors for n monolayers. FIG. 2B shows TEM image of ZnTe/3ZnSe heterostructures with unexpected dumbbell morphology. The size histogram indicates the average length of dumbbells is 16.2 nm with an average tip width of 6.3 nm (FIG. 3C), from which an average elongation of 2.1 nm along the rod direction can be extracted on both ends of ZnTe nanorods. The inset in FIG. 2B shows the geometric structure of ZnTe/3ZnSe NDBs. The absorption spectra of NDBs display a small peak ˜470 nm and a featureless tail ˜550 nm (FIG. 2C), corresponding to the absorption from ZnTe nanorods and the intermediate states at the junction between ZnTe and ZnSe respectively. The formation of alloyed nanoparticles is ruled out because the movement of anions is not efficient at the synthesis temperature (240° C.). The NDBs exhibit bright PL around 580 nm with a quantum yield (PL QY) of ˜18% at room temperature. ZnTe/ZnSe is a typical type-II structure, in which the holes are confined in ZnTe, whereas the electrons are localized in the ZnSe shell material according to the band alignments of bulk materials (FIG. 2D). The emission apparently originates from the radiative spatial indirect recombination of excitons of effective band gap determined by the valence band of ZnTe and the conduction band of ZnSe, because the emission energy is smaller than the band gap of either ZnTe or ZnSe.

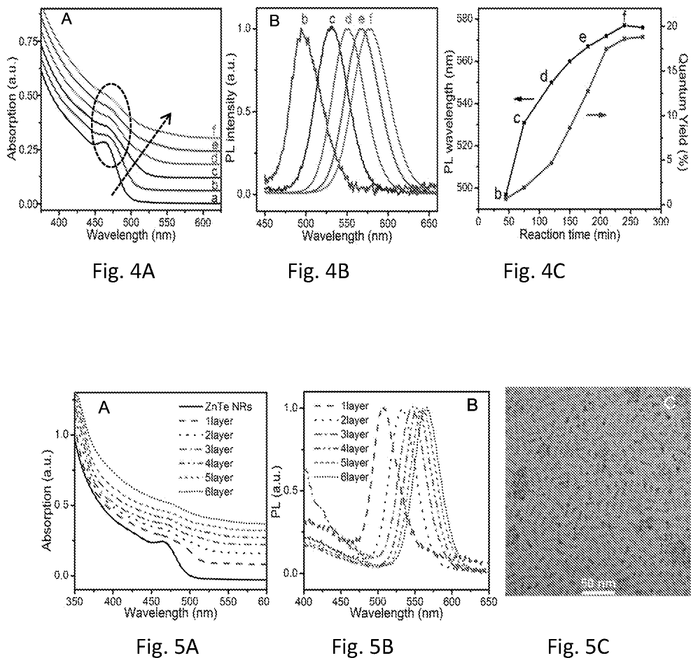

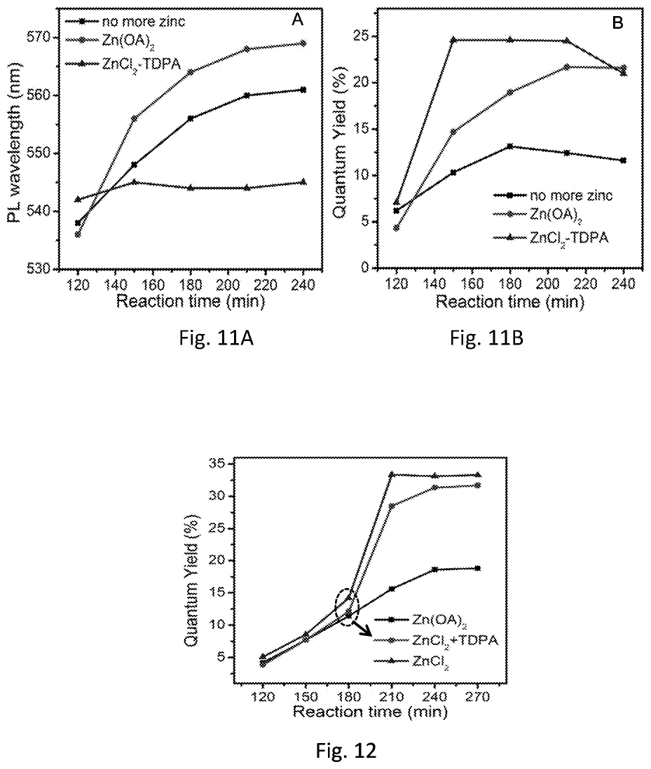

To monitor the progress of the ZnSe tip growth, the absorption and PL spectra were measured during the synthesis of ZnTe/3ZnSe as a function of reaction time (FIG. 4). After the first addition of Zn and Se precursors, a red shift of ˜7 nm of the excitonic peak (to ˜470 nm) is observed in the absorption spectrum. Due to the ZnSe growth, the nanoparticles becomes emissive (at ˜497 nm) as shown in FIG. 4B (b), although the quantum yield is low at this stage. As more Zn and Se precursors are introduced, the exciton peak of ZnTe can be seen during the whole synthesis (marked in the circle in FIG. 4A). This is consistent with the formation of NDBs structure. In typical type-II core/shell structure, a prominent red shift of excitonic absorption is expected as the shell grows. Meanwhile, a small absorption tail can be recognized and shifted to the red, indicating the decrease of the effective band gap upon the ZnSe tip growth (along the arrow in FIG. 4A). In this process, the emission wavelength continuously shifts to the red even when no more selenium precursor is added (from d to f in FIG. 4B and FIG. 11, black line), which means uncompleted consumption of added Se precursor between the addition internal time. Thereby, heating is continued until no more shifts are observed, during which more zinc oleate is added to promote ZnSe growth and improve the surface passivation. The obtained nanoparticles from adding additional zinc oleate display larger red shift of emission as well as higher PL QY (FIG. 11). The quantitative results of the PL wavelength and PL QY evolution are presented in FIG. 4C. The PL wavelength gradually red shifts from ˜500 nm to ˜580 nm, a typical feature for type-II structure. PLQY increases from less than 1% to ˜18% at the end of the growth process. ZnSe growth on ZnTe nanorods can also be performed by injecting selenium precursor (TOP-Se) in the solution of ZnTe nanorods dispersed in the mixture of TOP, oleylamine and zinc oleate at high temperature. A similar dumbbell structure, but less well defined, is obtained and PL QY is much lower than that via the above layer-by layer method (FIG. 5).

The formation of dumbbell structures is related to the high reactivity of rod end facets. The ZnSe nucleates favorably at the end of ZnTe nanorods instead of homogeneous nucleation, due to relatively low reactivity of ZnSe precursors at the synthesis temperature. This is evidenced by the absence of individual ZnSe nanoparticles in the synthesis.

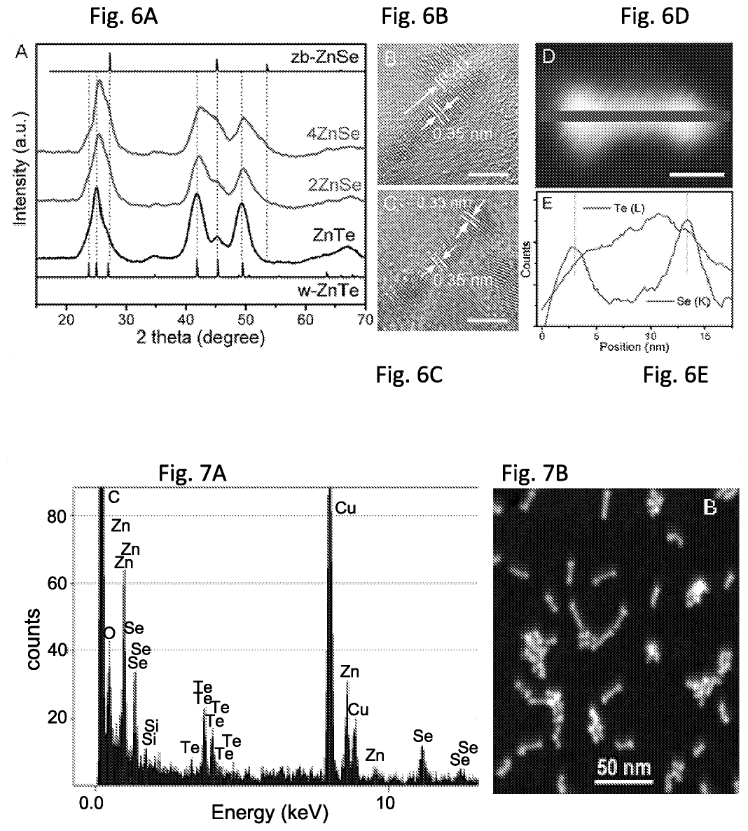

Powder X-ray diffractions (XRD) of bare ZnTe nanorods confirm the wurtzite structure of ZnTe as shown in FIG. 6A. The relatively sharp peak (002) at ˜25° indicates the favorable growth along the long axis, consistent with the above HRTEM analysis. The ZnSe growth induces this peak a very small shift to the high angle (<0.5°). Meanwhile, additional peaks (27.2°, 45.2° and 53.6°) can be recognized and become more intense as ZnSe grows. These additional peaks are indexed to zinc-blende ZnSe. The XRD reflections of ZnTe/ZnSe heterostructures can be considered as the ‘mixture’ of ZnTe and ZnSe, which further testifies the formation of ZnTe/ZnSe NDBs.

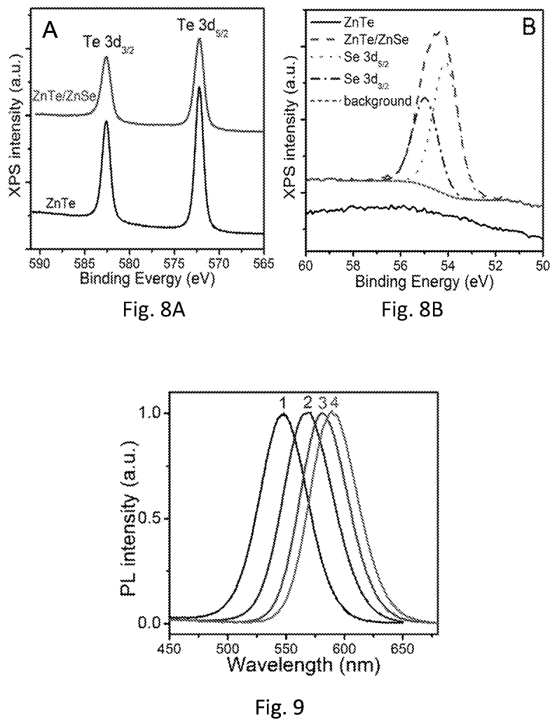

HRTEM images of ZnTe nanorods and ZnTe/3ZnSe NDBs are shown in FIG. 6B and C respectively. For ZnTe nanorods, HRTEM characterization reveals a 0.35 nm spacing of lattice fringes, which can be indexed to the (002) plane d-spacing of wurtzite ZnTe. It indicates ZnTe nanorods grow along [001] direction. ZnTe/ZnSe NDBs display a distinctively different morphology. In the middle part, a typical wurtzite structure with lattice fringes of 0.35 nm can be distinguished, demonstrating the maintained ZnTe nanorods. However, the two caps on both ends of ZnTe nanorod present a zinc-blende crystal structure, clearly different from the middle part. The measured lattice fringe of 0.33 nm is consistent with the (111) plane d-spacing of zinc-blende ZnSe Besides, stacking faults are clearly visible. Energy dispersive X-ray (EDX) measurement of an area shows the co-existence of Se and Te with comparative amounts (FIG. 7A). Scanning TEM (STEM) image also allows resolving the rod and the tips (FIG. 7B). The elemental analysis performed by a line-scan in STEM along the NDB indicates that the Se element is mainly distributed in the region of two tips whereas the Te element is mainly distributed in the nanorod area (FIGS. 6D, E). This result provides additional unambiguous confirmation for the dumbbell morphology of the ZnTe/ZnSe nanocrystals. Further support for the NDBs structure is provided by XPS, which is a surface sensitive technique. The Te 3d signals from ZnTe/ZnSe NDBs are not significantly screened by the ZnSe growth as expected for dumbbells structure, since ZnTe nanorods are not fully coated but rather the tip growth takes place (FIG. 8).

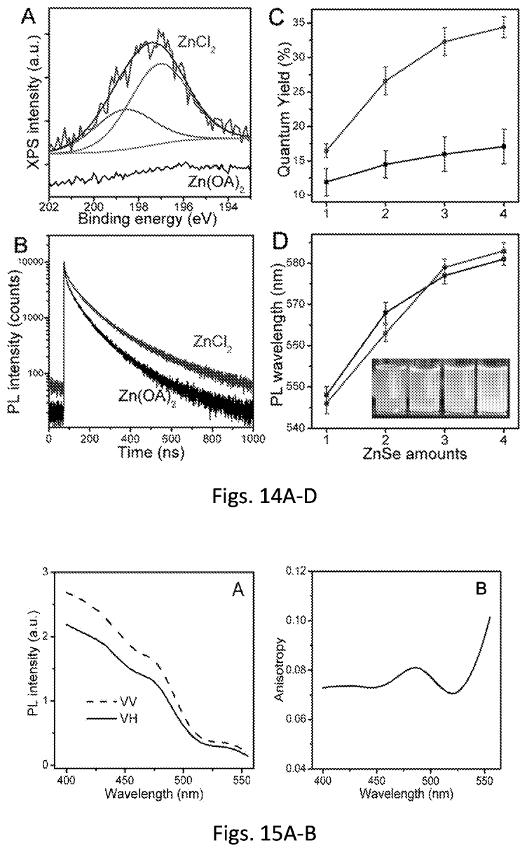

The emission control of the unique structure can be realized in one synthesis as performed in FIG. 4. However, PL QY in the middle of the synthesis is low (FIG. 4C). Thus ZnTe/ZnSe nanoparticles with tunable emission and high PL QY are obtained by adding different amounts of the ZnSe precursors via the layer-by-layer growth method. The reaction temperature is cooled down only after no more red shift of emission is observed. The emission spectra and quantitative results of PL wavelength and QY are shown in FIG. 9 and FIG. 14C, respectively. The emission wavelength ranges from ˜540 to ˜585 nm with the addition of ZnSe precursors with amounts equal to what would be expected for one to four monolayers coating the entire rod. The corresponding PLQY increases from ˜12% to ˜20%. TEM characterization indicates that the size of the ZnSe tips (the dimension perpendicular to c-axis of ZnTe nanorods) increases from ˜3.8 to ˜6.5 nm upon increased ZnSe amounts (FIG. 3). ZnTe/ZnSe NDBs are obtained and resolved when the amount of ZnSe precursors is greater than two monolayers equivalent. ICP-MS analysis shows all the samples are zinc-rich and the molar ratio of Se/Te increases as more ZnSe precursors are added (Table 2). The ZnSe tips sizes are calculated based on Se/Te ratios and in good agreement with the measured values.

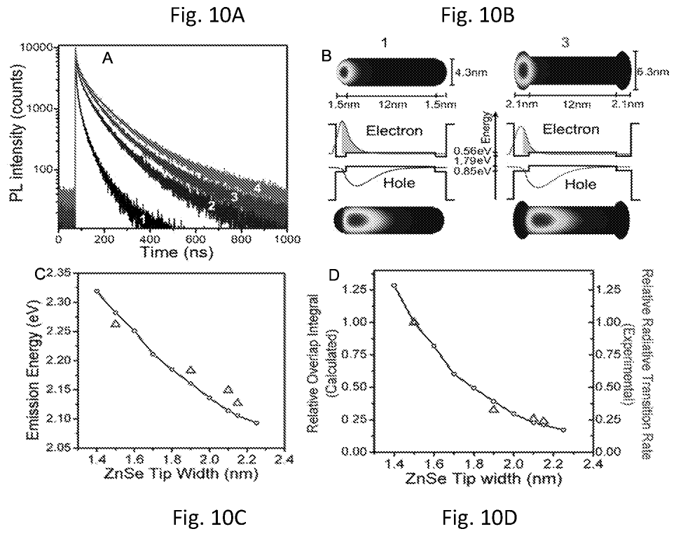

PL decays of these samples are shown in FIG. 10A. The measured lifetime is seen to increase upon growth of larger ZnSe tips, in general consistent with a development of a type-II junction. To be more quantitative—the effective lifetime (τ_{eff}) is defined as the time at which the PL intensity decreases to 1/e of the maximum value. Then the radiative lifetime can be estimated according to τ_{rad}=τ_{eff}/QY. Although this method has a limitation because it does not take into account the possibility of non-emitting particles, the estimation is nevertheless reasonable for the comparison of the different samples.

The transition from small to larger ZnSe tips leads to a change in the charge carriers distribution, which is manifested in the radiative lifetimes and in the exciton energy. In order to study the electronic structure of ZnTe/ZnSe NDBs, and in particular to probe the charge carriers' distribution throughout the nanoparticles, the self-consistent effective-mass Schrodinger-Poisson equations were solved numerically using Comsol Multiphysics module. Dimensions used for these simulations are based on the measured values, and using literature bulk parameters. FIG. 10B shows the band gap electron and hole wavefunctions for samples ZnTe/1ZnSe and ZnTe/3ZnSe in two representations. Note that in a strictly symmetric NDB of these materials and dimensions, the band gap transition is nearly two-fold degenerate and displayed only one of these states. In the actual NDBs this is the realistic situation considering the intrinsic differences between the two tips which will preferentially lead to one dominant lower exciton state. In any event, the single exciton will occupy one state.

TABLE 2
Relative concentrations of Zn, Se and Te measured by ICP-MS of ZnTe/ZnSe
NDBs with different amounts of ZnSe precursors as shown in FIG. 3 in the
main text. The measured and calculated ZnSe sizes of the dimension perpendicular
to c-axis of ZnTe nanorods are also shown. To simplify the calculation,
ZnSe tip is considered to a cylinder. The cylinder height ‘H’ is
extracted with the lengths of ZnTe nanorods and ZnTe/ZnSe NDBs. The volume of
ZnTe nanorod is considered as a constant. The ZnSe tip volume ‘V’ is then
obtained based on the Se/Te ratio and the volume of ZnTe nanorods. Then ZnSe
tip size ‘D’ is calculated by V = 2π × (D/2)² × H.

| | | | | | Measured ZnSe | Calculated ZnSe |
|---|---|---|---|---|---|---|
| | Zn | Se | Te | Se/Te | size from TEM | size from ICP |
| ZnTe/1ZnSe | 1 | 0.13 | 0.76 | 0.17 | 3.8 ± 0.3 nm | 4.2 nm |
| ZnTe/2ZnSe | 1 | 0.30 | 0.64 | 0.47 | 5.2 ± 0.4 nm | 5.3 nm |
| ZnTe/3ZnSe | 1 | 0.37 | 0.50 | 0.74 | 6.3 ± 0.4 nm | 6.4 nm |
| ZnTe/4ZnSe | 1 | 0.35 | 0.42 | 0.83 | 6.5 ± 0.5 nm | 6.5 nm |

The ZnSe tip size is found to be very important in determining the photophysical properties of these NDBs. Comparing samples 3 and 1, in the case of the larger tip (sample 3), the electron wavefunction is well localized in the tip leading to a smaller confinement energy and red shift of the band gap in comparison with sample 1. While in type-II systems the electron and hole are separated by the staggered potential profile, the coulombic binding energy attracts the hole towards the electron providing increased overlap between their wave functions with direct relation to the radiative lifetime. For the smaller ZnSe tips, the larger confinement energy of the electron leads to greater leakage of the electron wave function into the ZnTe nanorod region and hence to a larger electron-hole overlap as indicated by the gray shaded region of the electron wavefunctions in FIG. 10B. Correspondingly, these calculations predict an increase in the radiative rate as compared to the case of the larger tips.

Quantitative comparisons of the model calculations and the actual experimental data are presented in FIGS. 10C, D and Table 3. FIG. 10C shows the calculated PL emission energy (blue circles) of ZnTe/ZnSe NDBs as a function of ZnSe tip width along the C-axis of the ZnTe nanorod. Emission energies decrease as the size of the ZnSe tip increases, as expected. Experimental emission energies (marked as red triangles) are in good agreement with the simulated results. The calculated electron-hole overlap integrals |<Ψₑ|Ψₕ>|² as a function of ZnSe tip width is shown in FIG. 10D, using the overlap integral of ZnTe/1ZnSe NDBs as the reference. As the ZnSe tip size increases, the overlap between the electron and the hole rapidly decreases manifesting the localization of the electron to the tip while further increasing of the tip size decreases the overlap moderately as analyzed above. The measured relative radiative transition rate, extracted from the lifetime curves and QY data as explained above, which is proportional to the overlap integral |<Ψₑ|Ψₕ>|², also show the same behavior and is in a good agreement with the simulation.

As shown in FIG. 14C, PL QY of ZnTe/ZnSe NDBs increases as the tip size of ZnSe increases, despite decreasing overlap between the electron and hole wavefunction which is indeed manifested in the increased PL lifetime. The increased PL QY is assigned to decreasing the non-radiative decay rate caused by surface traps. Bare ZnTe nanorods suffer from extremely low QY because of the large amount of surface traps. The growth of ZnSe tips on the apexes of the ZnTe nanorod drives the electron wave function to localize on the tip. As demonstrated by the simulation, due to the coulombic interaction, the hole wave function is attracted by the electron wave function and is concentrated on the apex of the ZnTe nanorod near the ZnTe/ZnSe interface, which is properly passivated from surface traps. With increased coverage of the apex by larger ZnSe tips, surface hole traps are better passivated.

TABLE 3
A comparison between experimental and calculated PL wavelength
and corresponding measured radiative lifetime and calculated exciton
overlap of ZnTe with different amounts of ZnSe precursors.

| | Exp. | Calc. | Rad. | |
|---|---|---|---|---|
| | Wavelength | wavelength | Lifetime | |
| Sample | (nm) | (nm) | (nsec) | \|<Ψₑ\|Ψₕ>\|² |
| 1 | 548 | 543.4 | 31.1 | 0.18 |
| 2 | 568 | 573.7 | 95.6 | 0.07 |
| 3 | 577 | 586.4 | 121 | 0.04 |

To further increase the fluorescence quantum yield of these nanoparticles, a chloride treatment was applied to improve the optical properties of ZnTe/ZnSe NDBs. The chloride-contained solution is prepared by heating ZnCl₂, tetradecylphosphonic acid (TDPA), oleylamine and TOP at ˜100° C. under vacuum for 30 minutes. When this solution is added right after the last injection of selenium precursor, the red shift of PL wavelength is halted, which may be related to the strong complexion between Zn and TDPA that stops the ZnSe growth. Meanwhile, the PL QY was greatly enhanced from ˜5% to ˜25% (FIG. 11).

Thereby, the chloride treatment is performed at the end of synthesis and the temperature is maintained for 1 hour. Upon the chloride treatment, PL QY increases from ˜18% to more than 30% (FIG. 12). A control experiment is performed by using the same chloride-contained solution except no TDPA being added. In this case, similar PL QY increase is observed.

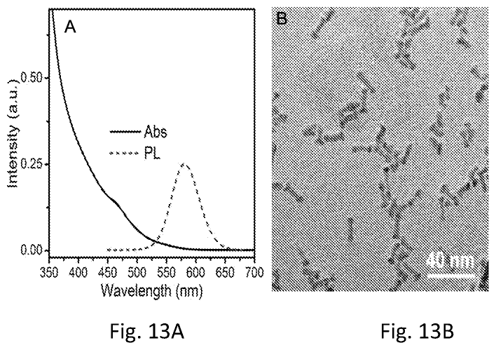

This result excludes the possibility that the strong complexation between zinc and phosphonic acid is responsible for the PL QY enhancement. The obtained ZnTe/ZnSe heterostructures with the chloride treatment display similar absorption and emission spectra as well as dumbbells shape (FIG. 13). FIGS. 14C and 14D shows the comparison of PL wavelength and PL QY of ZnTe/ZnSe NDBs with different amounts of ZnSe precursors without and with chloride treatment. The chloride treatment has little effect on PL wavelength, indicating the ZnSe growth is not altered. Meanwhile, all samples display a higher PL QY with the chloride treatment than their counterparts. The maximum of PL QY reaches ˜35%, an exceptional result for ZnTe based nanoparticles.

Examination of the presence of chloride on the surface of treated ZnTe/3ZnSe NDBs is given by XPS measurements (FIG. 14A). The Cl 2p XPS spectra of ZnTe/ZnSe NDBs without chloride treatment doesn't show any trace of chloride. With chloride treatment, a peak appears which can be fit to Cl 2p_{3/2} and Cl 2p_{1/2}, indicating the presence of chloride on the surface of ZnTe/ZnSe NDBs. The chloride treatment also increases the effective lifetime of ZnTe/3ZnSe NDBs (from 22.5 ns to 47.3 ns) as shown in FIG. 14B. A radiative lifetime of ˜140 ns is obtained, which is approximately equal to the radiative lifetime of ZnTe/3ZnSe NDBs without chloride treatment. This is reasonable since the chlorides mainly passivate the surface of NDBs. Based on these results, the mechanism suggested for the QY improvement is that the chloride only etches reactive surface selenium and/or tellurium atoms without changing the NDBs morphology. The chloride atoms on the surface decrease the surface traps, leading to a better surface passivation together with the original organic ligands, in accordance with the suggestions in previous studies.

The fluorescence of ZnTe/ZnSe NDBs is quenched very quickly when the solution is exposed to air. The quenching is caused by the oxidation of ZnTe. This is reasonable since the ZnTe nanorod part is not fully coated in the dumbbells structure obtained. The chloride treatment doesn't improve the stability of ZnTe/ZnSe NDBs in air.

A known property of nanorods is their linearly polarized absorption and emission. The emission polarization of ZnTe/ZnSe NDBs was also explored by using the excitation photo-selection method as proposed in the literature (FIG. 15). The sample of ZnTe/ZnSe NDBs dispersed in hexane (sealed in a cuvette) was excited by a vertical light, followed by the measurements of photoluminescence excitation (PLE) spectra parallel (I_{VV}) and perpendicular (I_{VH}) to the excitation light. The anisotropy was then extracted according to

$$r = \frac{{I}_{V V} - {I}_{V H}}{{I}_{V V} + 2 {I}_{V H}}$$

The ZnTe/ZnSe NDBs showed an anisotropy between 0.07 and 0.1 at the measured wavelength range, which was apparently lower than the most studied CdSe/CdS dot-in-rod or rod-in-rod systems, possibly because of the formation of NDBs instead of rod shaped core/shell structure. As discussed above, due to the staggered type-II band alignment between ZnTe and ZnSe, the holes were confined in ZnTe nanorods whereas the electrons were mainly localized in the ZnSe part. The emission originates from the radiative recombination of excitons across the interface of ZnTe and ZnSe.

### CONCLUSIONS

Colloidal heavy-metal-free type-II ZnTe/ZnSe NDBs are synthesized for the first time. The unique dumbbell morphology is confirmed by TEM, HRTEM, XRD and XPS measurements. The ZnSe growth makes these nanoparticles fluorescent, of which emission can be tuned from ˜500 nm to ˜585 nm by changing the tip size of ZnSe. PL QY can be greatly enhanced and reaches more than 30% with chloride treatment. Effective-mass based modeling shows that the hole wave function is spread over the ZnTe nanorods while the electron wave function is localized on the ZnSe tips. This is consistent with the relatively long lifetime of the obtained ZnTe/ZnSe NDBs, which is related to the type-II potential profile. The heavy-metal-free ZnTe/ZnSe NDBs show great potentials for the future display applications, lighting, lasing and more, especially when heavy-metal-contained materials are restricted.

1. A colloidal heavy-metal-free zinc chalcogenide nanostructure, the nanostructure comprising at least one elongated element of at least one zinc chalcogenide material, each of the at least one elongated elements having at least one tip ends coated with a heavy-metal-free semiconductor material, wherein the semiconductor material is different from the at least one zinc chalcogenide material.

2. The nanostructure according to claim 1, wherein the semiconductor material is a zinc chalcogenide material different from the at least one chalcogenide material.

3. The nanostructure according to claim 1, wherein each of the at least one tips is coated with a different semiconductor material.

4. The nanostructure according to claim 1, being a Type-II structure.

5. A Type-II heavy-metal-free zinc-based nanostructure, the nanostructure comprising an elongated element of at least one zinc chalcogenide having at least one tip ends, each of the tip ends being coated with a zinc-chalcogenide semiconductor material.

6. The nanostructure according to claim 1, wherein each of the tip ends is coated with a III-V semiconductor material.

7. The nanostructure according to claim 1, wherein the elongated element being or comprising a material selected from the group consisting of ZnTe, ZnSe, ZnS, ZnO and alloys thereof.

8. The nanostructure according to claim 1, wherein each of the elongated element tips is coated with a material selected from the group consisting of ZnSe, ZnO, ZnS, ZnTe, InN, GaN, InP, GaP, AlP and alloys thereof.

9. The nanostructure according to claim 1, being selected from the group consisting of ZnTe/ZnSe, ZnTe/InP, ZnSe/InP, ZnS/ZnSe, ZnS/ZnTe and ZnS/InP.

10. The nanostructure according to claim 1, being a nanorod coated on one or both of its end regions with at least one semiconductor material.

11. The nanostructure according to claim 10, wherein each end region is coated with a different semiconductor material.

12. The nanostructure according to claim 10, wherein the at least one semiconductor material is a zinc chalcogenide material being different from the material of the elongated element.

13. The nanostructure according to claim 10, wherein the length of the nanostructure is between 5 and 100 nm.

14. The nanostructure according to claim 13, wherein the average length of the nanostructure is between 5 and 90 nm, 5 and 80 nm, 5 and 70 nm, 5 and 60 nm, 5 and 50 nm, 5 and 40 nm, 5 and 30 nm, 5 and 20 nm, 10 and 90 nm, 10 and 80 nm, 10 and 70 nm, 10 and 60 nm, 10 and 50 nm, 10 and 40 nm, 10 and 30 nm or 10 and 20 nm.

15. The nanostructure according to claim 13, wherein the length is between 5 and 20 nm, 5 and 19 nm, 5 and 18 nm, 5 and 17 nm, 5 and 16 nm, 5 and 15 nm, 5 and 14 nm, 5 and 13 nm, 5 and 12 nm, 5 and 11 nm or 5 and 10 nm.

16. The nanostructure according to claim 13, wherein the length is between 6 and 20 nm, 6 and 19 nm, 6 and 18 nm, 6 and 17 nm, 6 and 16 nm, 10 and 20 nm, 10 and 19 nm, 10 and 18 nm, 10 and 17 nm, 10 and 16 nm, 10 and 15 nm, 15 and 20 nm, 15 and 25 nm, 15 and 30 nm or 15 and 35 nm.

17. The nanostructure according to claim 1, exhibiting tunable emission from ˜500 to ˜585 nm.

18. A method of tuning light emission from a zinc chalcogenide nanorod free of heavy metals, the method comprising forming or decorating the nanorod tips with at least one semiconductor material.

19. A device comprising a nanostructure according to claim 1.

20. The device according to claim 19, being an electronic device, an optical device, an optoelectronic device, a device used in medicine or a device used in diagnosis.

21. The device according to claim 19, being a display, a light conversion layer, a back light unit, a light emitting diode, or a sensor.
